(19) United States
(12) Patent Application Publication
(10) Pub. No.: US 2021/0275077 A1
(43) Pub. Date: Sep. 9, 2021

(54) DEVICE FOR MEASURING OR STIMULATING VITAL SIGNS OF A USER

(71) Applicants: WOLFGANG RICHTER, VANCOUVER (CA); FARANAK ZADEH, VANCOUVER (CA)

(72) Inventors: WOLFGANG RICHTER, VANCOUVER (CA); FARANAK ZADEH, VANCOUVER (CA)

(21) Appl. No.: 17/192,675

(22) Filed: Mar. 4, 2021

Related U.S. Application Data
(60) Provisional application No. 62/985,328, filed on Mar. 5, 2020.

(51) Int. Cl. A61B 5/268; A61B 5/00

(52) U.S. Cl. CPC A61B 5/268; A61B 5/0022; A61B 2562/0233; A61B 2562/08; A61B 5/685

(57) ABSTRACT

Disclosed is a novel hybrid of a device combined with independent integrated multi sensing automats that measures up to 64 different vital signs simultaneously and battery free(!). This device includes a glue-free suction patch, easy to attach to a patient's neck, wrists, temples (or selected body parts, if required). A base station contains electronic parts that provide a weak alternating electric field (AeF), which propagates over the patient's skin with no harm, to power and communicate with the suction patch. The electric field is bi-directionally data-modulated (proprietary phase-duplex) while remotely powering the chip at the same time. The suction patch gets a sequence from the base station to create desired vital signs sensors temporarily on-the-fly and sends the digitized sensing values radiation-free(!) continuously to the base station, which wirelessly connects to networks, computers, SmartDevices and applications.

### CROSS-REFERENCE TO RELATED APPLICATION

This application claims priority to U.S. Provisional Application No. 62/985,328, filed Mar. 5, 2020, which is incorporated herein by reference.

### BACKGROUND OF THE INVENTION

### 1. Field of the Invention

The present invention generally relates to a device for measuring vital signs of a user, and more particularly relates to a device for measuring vital signs of a user using a PolyMonIC patch.

### 2. Description of Related Art

Effective, efficient, and safe delivery of healthcare is dependent on the timely identification and treatment of a user's (also further called “patient”) condition. Failure to rescue patients in the early stage of physiological deterioration can result in permanent organ injury, extended medical treatment, increased recovery time, or death. These avoidable adverse events drive healthcare costs up and quality down.

Various medical devices are known in the art such as pulse oximeters featuring an optical module, typically worn on a patient's finger or ear lobe, and a processing module that analyzes data generated by the optical module. The optical module typically includes first and second light sources (e.g., light-emitting diodes, or LEDs) that transmit optical radiation at, respectively, red (λ\tilde{ }600-700 nm) and infrared ((λ\tilde{ }800-1200 nm) wavelengths. The optical module also features a photodetector that detects transmitted radiation that passes through an underlying artery within, e.g., the patient's finger or earlobe.

Another medical device called an electrocardiograph features conductive electrodes, placed at various locations on a patient's body, that measure electrical signals which pass into an amplification circuit. The circuit generates a waveform called an electrocardiogram, or ECG, that describes a time-dependent response of the patient's cardiovascular system.

Wireless vital signs sensors sending their data utilizing microwaves. Their radiations may be harmful for the user. Further, existing medical devices are complex to use and require wiring to be carefully placed onto the skin of the user. Therefore, there is a need for a device for measuring vital signs of a user which is preferably radiation free and works wirelessly. Further, the device should be able to stimulate the vital signs of the user.

### SUMMARY OF THE INVENTION

In accordance with teachings of the present invention, a device for measuring vital signs of a user is provided.

An object of the present invention is to provide a device including a polymer patch attached to the user, a PolyMonIC chip is embedded inside the polymer patch to measure vital signs data from the vital signs of the user, and a base station is coupled to the PolyMonIC chip to communicate the received vital signs data over the communication network.

The base station generates commands and emits a modulated alternating electric field. Further, the base station demodulates the vital signs data received from the PolyMonIC chip. The PolyMonIC chip converts the vital signs data into digitized results and communicates back to the base station via the modulated alternating electric field.

Another object of the present invention is to provide the device with optoelectronic blocks arranged to perform measurements of the vital signs of the user using optical effects, and a plurality of piezo rings arranged inside the polymer patch to convert mechanical vital signs into electrical signals.

Another object of the present invention is to provide the device wherein the piezo rings are arranged inside the polymer patch to convert electrical pulses from the PolyMonIC chip into mechanical signals to stimulate the user.

Another object of the present invention is to provide the device with an electronic Ultra-Turing machine to alter a sequence in a digital unit according to digitized calculation results of the processed vital signs to optimize the performance of the digital unit, and a superposition unit, containing a Pascal triangle structure, for calculating a dynamic baseline for drift compensation and probabilities to classify the received processed vital signs.

Another object of the present invention is to provide the device with a unique identifier to generate a unique identification code for identifying the user, and a plurality of micro-needles arranged within the polymer patch to penetrate the skin to apply negative/positive charges, bio-chemical substances etc.

Another object of the present invention is to provide the device with a plurality of reservoirs arranged to store bio-chemical substances to be released into the user through the micro-needles. Further, the plurality of micro-needles act under the control of integrated ionizer circuits.

Another object of the present invention is to provide the base station with an interface for communicating over communication networks. Further, the PolyMonIC chip includes an optical unit attached to the polymer patch for generating signals based on the vital conditions of the user, and a pin hole to guide the optical effects to and from the optoelectronic blocks.

While a number of features are described herein with respect to embodiments of the inventions; features described with respect to a given embodiment also may be employed in connection with other embodiments. The following description and the annexed drawings set forth certain illustrative embodiments of the inventions. These embodiments are indicative, however, of but a few of the various ways in which the principles of the inventions may be employed. Other objects, advantages, and novel features according to aspects of the inventions will become apparent from the following detailed description when considered in conjunction with the drawings.

### BRIEF DESCRIPTION OF DRAWINGS

The annexed drawings, which are not necessarily to scale, show various aspects of the inventions in which similar reference numerals are used to indicate the same or similar parts in the various views.

FIG. 1 illustrates a schematic diagram of the device for measuring and stimulating vital signs of a user;

FIG. 2 illustrates a schematic diagram of the PolyMonIC chip;

FIG. 3 illustrates a schematic diagram of a SuRe super-register Block; and

FIG. 4 illustrates a cut-out of the polymer patch and PolyMonIC chip to showcase arrangement of electronic components.

### DETAILED DESCRIPTION OF DRAWINGS

The present disclosure is now described in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

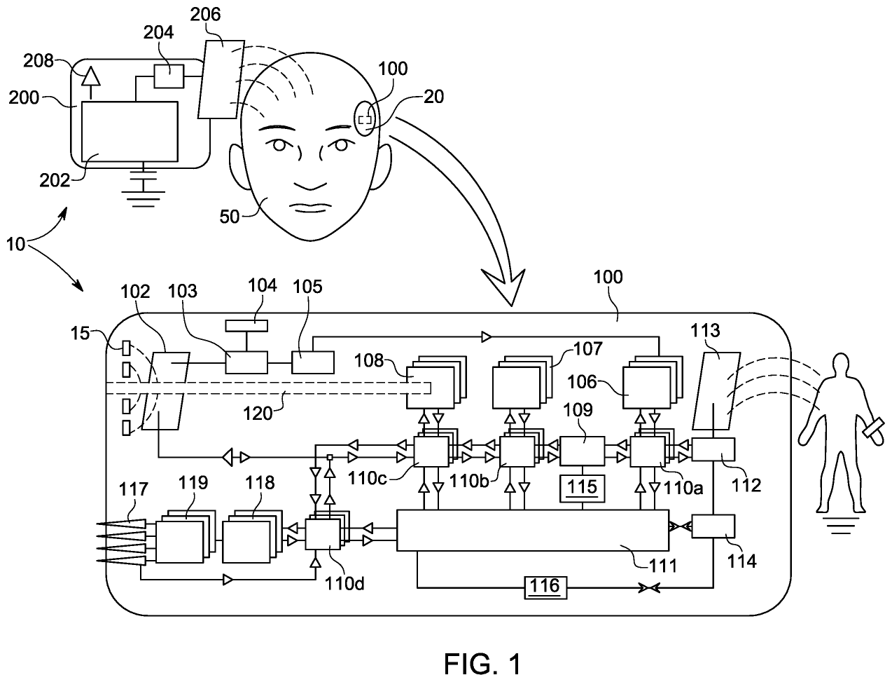

FIG. 1 illustrates a schematic diagram of the device 10 for measuring and stimulating vital signs of a user 50. The device 10 includes a polymer patch 20 attached to the user 50, a PolyMonIC chip 100 embedded inside the polymer patch 20 to measure vital signs data from the vital signs of the user 50, and a base station 200 capacitively coupled to the PolyMonIC chip 100 to communicate the received vital signs data over the communication network.

In a preferred embodiment of the present invention, the base station 200 includes a controller 202, a convertor 204, and a first electrode 206. The controller 202 generates a frequency and commands for the PolyMonIC chip 100. The convertor 204 filters and modulates the frequency. Further, the convertor 204 demodulates the processed vital signs data received from the PolyMonIC chip 100.

The first electrode 206 emits the modulating frequency as an alternating electric field for influencing the user 50. Examples of the controller 202 include but not limited to MCU, logic circuits, FPGA, SoC etc. Examples of the convertor 204 include but not limited to a mixer, resonator, analog multiplier, IQ-(de-) modulator, etc.

In another preferred embodiment of the present invention, the base station 200 includes an interface 208 for communicating processed data received from the controller 204 over communication networks. Further, the interface 208 receives instructions over the communication network to be communicated with the controller 202.

Examples of the interface 208 include but not limited to serial, USB, Wi-Fi, Bluetooth, GSM, CDMA, LTE, and any other communication devices. Further in another embodiment of the present invention, the interface 208 further communicates with interfaces of other base stations from other polymer patches to share vital sign data among users.

The user may be a human, animal or plant. The device works with the polymer patch either attached, near or placed inside the body of the user. The base station is a microcontroller (ARM MCU). One of its output pins provides a pulse-width modulated rectangle signal ˜500 kHz which is connected to a flexible electrode via a resonator fork. This results in a weak alternating electric field (AeF) emitted from the electrode.

The base station easily connects to routers, wireless networks, and smart devices (and can use their GPS features for cost savings). Adequate I2C peripheral modules like display, speaker, SD data storage or GPS are optionally available. The MCU is programmed e.g. in Python with a novel A.I. (3.0) core for virtual assistance (VPA).

The controller 200 may be programmed with a “virtual personal assistant” (VPA) A.I. machine learning software that is trained to react to a patient's vital state. The base station (also called Bi-O-watch) may be placed, or attached near the patient, e.g. on a bed, stretcher or (wheel-)chair. Yet, it may be worn by the patient or a caretaker to be mobile like a SmartWatch, hence the name.

In a preferred embodiment of the present invention, the PolyMonIC chip 100 includes a bifilar electrode 102, a second convertor 103, a buffer 104, a sub-circuit 105, plurality of digital electronic blocks 106, plurality of analog electronic blocks 107, a third convertor 109, plurality of electronic switches 110, a digital unit 111, a bi-directional communication unit 112, and a second electrode 113. The PolyMonIC chip 100 contributes to the groups of “flexible hybrid electronics” FHE, flexible, or printed electronics PE.

The bifilar electrode 102 receives the alternating electric field and the electric vital signs from the user 50. The second convertor 103 rectifies and converts the alternating electric field into DC energy. The buffer 104 stores the DC energy in the polymer patch 100. The sub-circuit 105 extracts clock signals from the alternating electric field frequency. The plurality of analog electronic blocks 107 are arranged to process the received vital sign data into analog values. The third convertor 109 converts the analog values into digital values and vice versa.

The plurality of digital electronic blocks 106 are arranged to perform logical operations on the digital values to generate a digitized result. The plurality of electronic switches 110 control the operation of the analog electronic blocks 107, the plurality of digital electronic blocks 106 and the other electronic components of the PolyMonIC chip 100.

The digital unit 111 sequences vital signs values between the analog electronic blocks 107 and the digital electronic blocks 106 utilizing the plurality of electronic switches 110a, 110b, respectively. The electronic switches 110a control the operation of the digital electronic blocks 106, and the electronic switches 110b control the operation of the analog electronic blocks 107.

The bi-directional communication unit 112 receives commands from the base station 200. The bi-directional communication unit 112 further modulates the processed digitized results received from the plurality of digital electronic blocks 106 into the alternating electric field to be demodulated from the convertor 204. The second electrode 113 closes the circuit with the base station 200, the user 50 and the earth's ground.

Examples of the second convertor 103 include but not limited to a rectifier, MOSFET switches, cascades, filters etc. Examples of the buffer 104 include but not limited to a capacitor, polymer structures, accumulators, silicon-/lithium-/chemical-/printed-batteries etc. Examples of the sub-circuit 105 include but not limited to Schmitt trigger, comparator, operational amplifier etc.

Examples of the digital electronic blocks 106 include but not limited to logic gates, registers, ALU, digital shifters, digital pointers and calculators etc. Examples of the analog electronic blocks 107 include but not limited to amplifiers, filters, oscillators, S&H, INA, multiplier, references etc.

Examples of the third convertor 109 include but not limited to ADC, DAC, comparators, perceptrons, etc. Examples of the electronic switches 110 include but not limited to transmission gates, MOSFET switches, etc. and examples of the digital unit 111 include but not limited to a memory pointer/counter combination, array of shift registers etc. Examples of the bi-directional communication unit 112 include but not limited to a transistor, transmission gates, CMOS, FET switches etc.

In another preferred embodiment of the present invention, the PolyMonIC chip 100 includes plurality of optoelectronic blocks 108 arranged to perform measurements of the vital signs of the user using optical effects. The optoelectronic blocks 108 are connected to the analog electronic blocks and the digital electronic blocks for further processing of the optical effects under the control of the digital unit 111.

Further, the electronic switches 110c control the operation of the optoelectronic blocks 108 under the control of the digital unit 111. Examples of the optoelectronic blocks 108 include but not limited to light emitting diodes, photosensitive semiconductors, optical filters, transimpedance amplifiers, etc.

In another preferred embodiment of the present invention, the device 10 further includes a plurality of piezo rings 15 arranged inside the polymer patch 20 to convert mechanical vital signs into electrical signals capacitively coupled to and received from the bifilar electrode 102.

Further, the plurality of piezo rings 15 arranged inside the polymer patch 20 convert electrical pulses from the PolyMonIC chip 100 into mechanical signals to simulate the user 50. Examples and variations of the piezo rings 15 include but not limited to crystals, FSR, polymer, electrostatic transducers etc.

In another embodiment of the present invention, the PolyMonIC chip 100 further includes an Ultra-Turing machine 114 to alter the sequence in the digital unit 111 according to digitized calculation results of the processed vital signs to optimize the performance of the digital unit 111, and a superposition unit 115 containing a Pascal triangle structure for calculating a dynamic baseline for drift compensation and probabilities to classify the received processed vital signs.

Examples of the Ultra-Turing machine 114 constitute of components including but not limited to arrangement of registers, counters, and hardware command interpreters, etc. Examples of the superposition unit 115 constitute of components including but not limited to a digital Pascal triangle, arrangement of perceptrons, neural networks, machine learning elements, digital slide rulers, counters, pointers etc.

In another preferred embodiment of the present invention, the PolyMonIC chip 100 further includes a unique identifier 116 to generate a unique identification code to be received by the base station 200 on demand modulated by the bi-directional communication unit 112. Examples of the unique identifier 116 include but not limited to a digital accessible array of numbers, character or symbols etc. Examples of the unique identification code include but not limited to a numeric code, alphabets code, alpha-numeric code, special character code, symbols, hashtags, etc. or in combination.

In another preferred embodiment of the present invention, the device 10 further includes plurality of micro-needles 117 arranged within the polymer patch 20 to penetrate the human skin, and plurality of ionizer circuits 118 arranged to perform positive and/or negative charges under the control of the digital unit 111 on either the bifilar electrode 102 or the plurality of micro-needles 117.

Examples of the micro-needles 117 include but not limited to bio-needles, micro-tubes, capillary-tubes, Nano tubes, micro syringes, micro-injectors, micro extruders, etc. In a preferred embodiment, the micro-needles 117 are bio-needles that dissolve in the user's skin. Examples of the ionizer circuits 118 include but not limited to anion/cation cascades, rectifiers, face controlled switches, etc.

In another preferred embodiment of the present invention, the device 10 further includes a plurality of reservoirs 119 arranged to store bio-chemical substances to be released into the user 50 through the micro-needles 117. The reservoirs 119 are made up of micro tanks, bubbles, liquefiable solutes, etc.

Further, the plurality of reservoirs 119 arranged to store bio-chemical substances to be released into the user 50 through the micro-needles under the control of the ionizer circuits. The ionizer circuits 118 are controlled by the electronic switches 110d under the control of the digital unit 111.

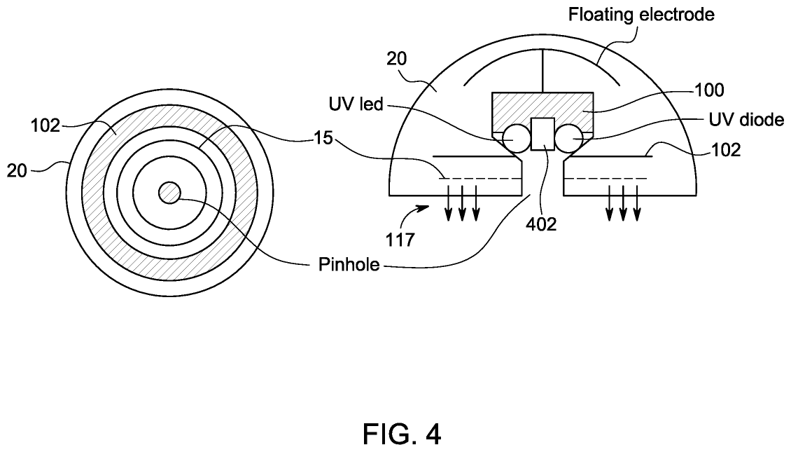

In another embodiment of the present invention, the device 10 further includes an optical unit (shown in FIG. 4) attached to the polymer patch 20 to generate signals based on vital conditions of the user 50. Examples of the optical unit include but not limited to a LED light, LCD light, OLED light etc.

In another embodiment of the present invention, the device 10 further includes a pin hole 120 to guide the optical effects to and from the optoelectronic blocks 108. Examples of the pin-hole include but not limited to tubes for receiving capillary or micro-fluidic provided samples for optical tests.

Every vital sign parameter is measured in at least two different ways to increase confidence in the results. The patient or a helper places the PolyMonIC chip either over the carotid artery on the neck or on the temple or on the wrist pulse. Any other position would focus on the myogram of the muscle below the patch which could be of great help to support disabled or elderly people in their activities (ECG and pneumo may still be detected).

Multiple patches may be used to optimize physio-therapeutics or fitness training. Also, animals (e.g. livestock, pets, etc.) Plants (for food, recreation, air quality, etc.) may be observed and/or stimulated if said PolyMonIC chips are attached in one or more polymer patches.

The firmware for the Bi-O-watch base station introduces a novel system of “virtual assistance” (VPA). A “virtual patch assistant” takes care of the sensing sequences and the proper function of a PolyMonIC patch. Another VPA, the “virtual personal assistant” takes care of a patient's existential orientation, mental state and wellbeing. A “virtual performance assistant” controls the sensing results and compares them with norm ranges while communicating with applications, computers and networks.

VPAs interpret scripts, which can be entered via a secure browser, e.g. via a HTML instructor page. All measurement data is encrypted, stored in a blockchain-like secure database which can be remotely accessed by authorized observers. They also will be automatically alerted if vital signs are out of selected range. As a pure numeric system, no personal data can be extracted or misused.

The Bi-O-watch base station may sense and interpret movements from about ˜5 m distance to record the activeness of the patient and caretakers, or other persons, wearing PolyMonIC chips (e.g. to ensure or support physical distancing in case of viral spreading pandemics). Medical devices, syringes and medication boxes could have PolyMonIC chips attached (e.g. on labels) which helps to prevent misuse or treatment errors.

Further, the Bi-O-watch base station contains a list of first responders and messages them whenever necessary (e.g. critical situations). As an example, a PolyMonIC may be attached on a person's belly to check the bladder to prevent urination in case of incontinence.

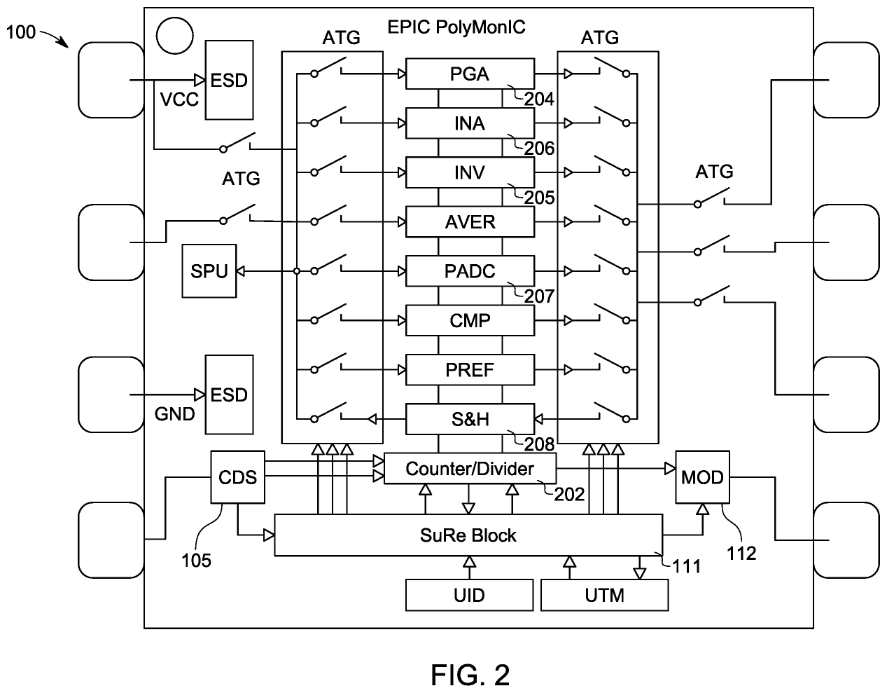

FIG. 2 illustrates a schematic diagram of a coin-sized PolyMonIC chip 100 measuring vital signs of the user. The PolyMonIC chip 100 consists of a tiny (e.g. <1 mm²) integrated silicon circuit. The PolyMonIC chip 100 contains routable optoelectronic-, analog-, and digital sub-circuits as building blocks for various biomedical sensors. One of those digital blocks is a sequencer (digital unit) 111 connected to a data transceiver block (bi-directional communication network) 112, that capacitively (non-magnetic) bi-directional communicates with an A.I. base station (called Bi-O-watch) in reach of the user. The base station provides an alternating electric field (AeF).

A user (patient) in reach (<1 m) couples capacitively with the alternating electric field (AeF), which propagates over the complete skin (dermis) with no harm. The patient wears a coin-sized conductive silicone rubber patch with an embedded PolyMonIC chip, which also is influenced by the alternating electric field (AeF).

The PolyMonIC chip has an integrated harvester (same as second convertor 103, shown in FIG. 1) that converts the alternating electric field (AeF) into DC energy (˜1 mW) and a precise field-synchronized operation (system-) clock for its sub-circuits. The MCU (base station) sends a sensor configuration sequence e.g. by modulating the duty cycle or phase of the alternating electric field (AeF) frequency, which is securely received by the PolyMonIC's data transceiver block (bi-directional communication unit 112 or modulator (MOD)) and linked to the sequencer 111 (SuRe Block/Digital Unit).

Thus, the sequencer 111 connects the required sub-circuits via transmission gates to build e.g. a temperature, SpO2, blood pulse & pressure-, or any other required sensor. The sensor configuration stays stable until a new sequence is received. The PolyMonIC chip 100 also measures physical forces or chemical reactions near, on, or in objects, items, liquids, and gases.

The sensing results are repeatedly encrypted as a payload and, together with a unique identifier (ID), sent back from the data transceiver block to the Bi-O-watch base station by modulating the alternating electric field (AeF) via a subcarrier, derived from the system clock. In this case, the conductive material of the patch acts as an electrode, yet it can also receive vital signs such as myograms from the respiratory (and other) muscles and the heart (ECG) or the brain (EEG), which evidentially propagate over the patient's dermis.

A PolyMonIC chip resembles a “biomedical laboratory on chip”; all necessary components are integrated to perform the required vital signs sensing (and beyond). The chip is self-calibrating, self-powered, and under a permanent control of at least one Bi-O-watch base station.

The PolyMonIC chip is e.g. embedded in a round (coin sized) dielectric polymer, which serves as a linear PTC temperature-impedance sensor as well as a glue-free suction patch to stick comfortably on the patient's skin. On the bottom, the PolyMonIC chip has a pinhole centered in the middle, surrounded by concentric rings printed from highly pressure-sensitive material (like piezo, Velostat, FSR, etc.). Further, the PolyMonIC chip may be equipped with special coatings or micro needles (shown in FIG. 1), e.g. to controllable release stimulants or drugs into the user's organism.

Another insulated conductive layer serves as an alternating electric field (AeF) electrode 102. It has a bifilar structure to resist magnetic influence (e.g. when used during MRI). The pressure-sensitive (piezo) rings grant proper functionality when attached e.g. over the artery, irrespective of orientation. They simultaneously detect the pulse wave, blood speed, and blood pressure by creating an analog voltage that is, preferably capacitively-coupled, received by the patch's electrode 102 for further processing from the PolyMonIC chip.

Due to the number of piezo rings (15, as shown in FIG. 1 and FIG. 4), this allows a far more accurate and comfortable blood pressure measurement than with a single-point tonometer. In a special application the piezo rings may vibrate under the control of the PolyMonIC chip's sub-circuits with variable frequencies, e.g. to create stimulating sounds, our pattern.

Further, the piezo rings may act as a bone-conductive transducer to receive audio signals from or over the user to be processed from at least one of the PolyMonIC chip, the Bi-O-Watch base station, or connected computers or networks. Additionally, through the pinhole, the PolyMonIC's optoelectronic sub-circuits may measure pulse, IR infrared temperature, UV SpO2 oxygen saturation, photoplethysmography, even detecting i.e. UV-A radiation, skin problems (e.g. mycosis), or pathogens.

In a preferred embodiment, the PolyMonIC chip 100 is designed in 28 nm ultra-low power mixed signal technology. While the energy harvester (second convertor 103, shown in FIG. 1) may provide up to 5 mW, the internal consumption is less than 1 mW (if the LEDs are used), at around 100 uW on average. The harvester ( ) also provides up to 5 kV ESD protection.

A Schmitt trigger 105 (sub-circuit 105, shown in FIG. 1) creates a rectangle system clock from the alternating electric field (AeF). A system counter block 202 (part of digital block 106, shown in FIG. 1) derives all necessary timing for the subcircuits. While not required in the PolyMonIC chip 100, the harvester (second converter 103, shown in FIG. 1) may charge connected (e.g. printed) batteries, just to be even more versatile.

The optoelectronic sub-circuits consist of RGB, IR, and UV LED blocks, RGB, IR, and UV photodiodes, a driver block and a sensitive (around 1 nA) transimpedance amplifier. Other analog components (107, as shown in FIG. 1) integrated in the PolyMonIC chips are programmable gain amplifiers (PGA) 204, a shield buffer (INV (inverter OPamp)) 205, an instrumentation amplifier (INA) 206, as well as a two stage Sample & Hold FET buffer (S&H) 208. An NTC diode (PADC) 207 as a third component may measure temperature accurately.

The PGA 204 is routable under the control of the sequencer (SuRe Block) 111 to act like a summing-, differential-, integrator/differentiator amplifier, or as a filter with variable gain in switched capacitor technology. All components have compensated transmission gates (ATG) (in NMOS/PMOS technology, also termed as electronic switches 110a,b,c,d in FIG. 1) on their in- and outputs, which are controlled by the sequencer block 111.

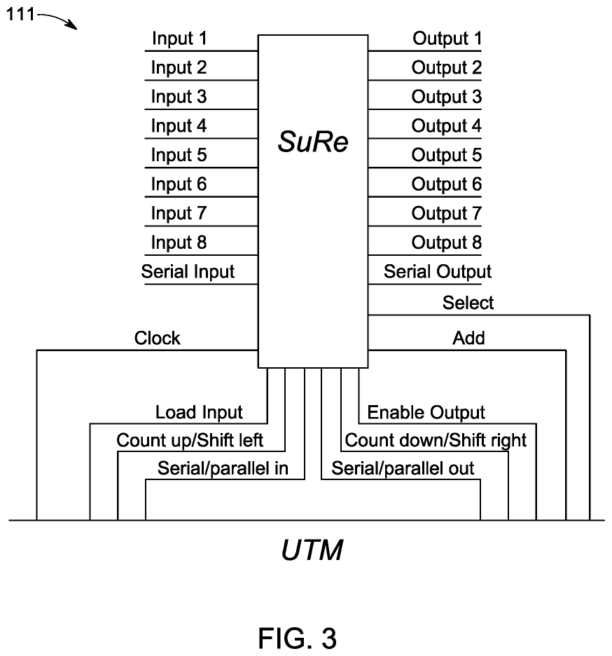

FIG. 3 illustrates a schematic diagram of a single SuRe Block 111 to serve in an arrangement of super registers. In a preferred embodiment of the present invention, the PolyMonIC chip (100, shown in FIG. 1 and FIG. 2) digital block contains e.g. 16 “super registers” (SuRe).

It would be readily apparent to those skilled in the art that different numbers of super registers 111 may be envisioned without deviating from the scope of the present invention. Each SuRe contains a serial/parallel input/output shift register. SuRe may turn into an up/down counter or process logical operations with the ICU and is fully controllable by the UTM (114, shown in FIG. 1 and FIG. 2).

The UTM ultra-Turing machine may be a self-propelling unit of e.g. 128 bytes which runs at gate speed (typically <2 ns/operation). The UTM only needs 16 commands (4 bit) to perform any logical or mathematical task, typically provided by the sequencer block. It would be readily apparent to those skilled in the art that different numbers of bytes and commands may be envisioned without deviating from the scope of the present invention.

The unique SPU block (115, shown in FIG. 1) performs Bayes' probability—as well as baseline calculation and drift compensation for the sensors. This superposition unit SPU emulates a mechanical slide ruler utilizing a combination of shift-registers (which resemble the movable sliders and tables of such a ruler). Pointers (digital counters 106, shown in FIG. 1) resemble the markers to point to the desired results. The SPU may also resemble a Pascal triangle.

The SuRe block 111 has multiple input and output connections e.g. as shown in FIG. 3, 8 inputs and 8 outputs. The SuRe block may convert serial signals into parallel and vice versa, add or subtract digital values, drive clocks, and performs logical or arithmetic shift operations.

FIG. 4 illustrates a cut-out of the polymer patch and PolyMonIC chip 100 to showcase the arrangement of electronic components. The piezo rings 15 of the PolyMonIC chip also act as a crystal microphone which listens to the patient while the polymer of the patch attenuates and reduces outside noise.

The bifilar conductive electrode 102 is insulated in the polymer patch 20. In a preferred embodiment the number of piezo rings 15 are 3. However, it would be readily apparent to those skilled in the art that various numbers of piezo rings may be envisioned without deviating from the scope of the present invention.

Examples of the optoelectronic block (108, shown in FIG. 1) includes but not limited to a IR receiver thermometer 402. Further, the FIG. 4 shows a plurality of micro-needles 117 attached to the bottom of the polymer patch 20 to be inserted/penetrate into a user's skin.

The device 10 is run on (wirelessly rechargeable) batteries, a Bi-O-watch base station may operate ˜10 days, yet it also may operate with every 5V cell phone charger connected. It also will be available as a battery-free NFC sticker! A PolyMonIC chip is not only designed for sick people. Health care starts with prevention, and vital signs monitoring is crucial to save our fellow citizens from malaise.

PolyMonIC chips also prevent accidents because they can warn their users when they enter dangerous zones. Disabled and elderly people will have a higher quality of life and more acceptance in the workplace. Last but not least, PolyMonIC & Bi-O-watch is a template for the world to make health care available for everybody, while saving enormous costs. Brands can use the patches as desired marketing (advertising) instrument while contributing to public health.
Typical usage of the present invention:

Analog audio signals from the patient are always be transmitted to the Bi-O-watch base station. Vital signs like Coughing, sneezing, vomiting or snoring may be identified by the base station's A.I., as well as spoken simple commands (e.g. “Help”, “Call Doctor”, “Status”, “Light”, etc.). The AI's VPAs perform sensing and inform the right responders.

The Bi-O-watch base station may also be used to control appliances for assisted living (e.g. ageing-at-home) or (autonomous) wheelchairs, or as “digital assistants” in the workplace as a novel frontend for edge computing and “ambient intelligence”.

With a built-in real-time clock of highest accuracy (e.g. 2 us), the Bi-O-watch records all relevant changes in the vital signs with a timestamp. It specially monitors the sleep cycle of a patient and calculates indisposition trends (e.g. flu symptoms increase/decrease, apnea, sweating, fever, etc.). This allows to keep distance e.g. to avoid viral spread or other types of contamination.

If the Bi-O-watch base station is equipped with a display and/or speaker, the patient may ask for the vital signs' status and get a prompt answer. As a variation, the PolyMonIC patch may be illuminated (RGB) which can be useful to enhance its acceptance as a wearable device, for cosmetic reasons or in group sessions or just for simpler visual inspection (e.g. fever).

The polymer patch also serves as a single active electrode to receive vital signs such as ECG signals from the heart (or EEG from brain or nerves), and muscle myograms of limbs, eyes, larynx, lungs, propagating over the human skin. As signals weaken over distance, it is apparent to place a patch close to the point of interest (e.g. sternum to check ECG and breathing) where they at least attenuated. The PolyMonIC chips also detect vital signs of users such as motion, reflexes, impacts on joints, jumping, falling, coughing fit, etc.

Apnea is a life critical disorder affecting the youngest and the elderly. A reliable “always on” sensor system like described would be of great help. The PolyMonIC chips are also ideal as a fitness companion during sports and training, with the Bi-O-watch base station as a competent “coach”. In a special application the invention related PolyMonIC chips may monitor and stimulate food plants grow or livestock, or may measure and enhance CO2 conversion in trees, algees, etc.

The present invention offers various advantages such as providing a device that measures vital signs wirelessly and without the usage of radio waves. Further, the device as an implanted chip costs only less than 10 cents. Further, the device may be attached with other similar devices for allowing collision-free 24/7 “always-on” observation for patients/users. The PolyMonIC patches are hacker-safe, require no programming (=bug-free), and accept only sequences from the Bi-O-watch base station. The weak alternating electric field (AeF) only works in a range of about -2m and forms a kind of “synthetic aura” around a person.

It should be appreciated that many of the elements discussed in this specification may be implemented in a hardware circuit(s), a circuitry executing software code or instructions which are encoded within computer readable media accessible to the circuitry, or a combination of a hardware circuit(s) and a circuitry or control block of an integrated circuit executing machine readable code encoded within a computer readable media. As such, the term circuit, module, server, application, or other equivalent description of an element as used throughout this specification is, unless otherwise indicated, intended to encompass a hardware circuit (whether discrete elements or an integrated circuit block), a circuitry or control block executing code encoded in a computer readable media, or a combination of a hardware circuit(s) and a circuitry and/or control block executing such code.

All ranges and ratio limits disclosed in the specification and claims may be combined in any manner. Unless specifically stated otherwise, references to “a,” “an,” and/or “the” may include one or more than one, and that reference to an item in the singular may also include the item in the plural.

Although the inventions have been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the inventions. In addition, while a particular feature of the inventions may have been described above with respect to only one or more of several illustrated embodiments, such feature may be combined with one or more other features of the other embodiments, as may be desired and advantageous for any given or particular application.

1. A device for measuring vital signs of a user, the device comprising:
a polymer patch attached to the user;
a PolyMonIC chip embedded inside the polymer patch to measure vital signs data from the vital signs of the user; and
a base station coupled to the PolymonIC chip to communicate the received vital signs data over the communication network, the base station comprising:
a controller to generate a frequency and commands for the PolymonIC chip;
a convertor to filter the frequency and further the convertor modulates the frequency with the commands, further the convertor demodulates the vital signs data received from the PolymonIC chip;
a first electrode for emitting the modulating frequency as an alternating electric field for influencing the user;
the PolyMonIC chip comprising:
a bifilar electrode to receive the alternating electric field and the electrical vital signs from the user;
a second convertor to rectify the alternating electric field and convert into a DC energy;
a buffer to store the DC energy in the polymer patch;
a sub-circuit to extract clock signals from the alternating electric field frequency;
a plurality of analog electronic blocks arranged to process the received vital sign data into analog values;
a third convertor for converting the analog values into digital values and vice versa;
a plurality of digital electronic blocks arranged to perform logical operations on the digital values to generate a digitized result;
a plurality of electronic switches to control the operation of the analog electronic blocks, the plurality of digital blocks and the other electronic components of the PolyMonIC chip;
a digital unit for sequencing vital signs values between the analog electronic blocks and the digital electronic blocks utilizing the plurality of electronic switches;
a bi-directional communication unit for receiving commands from the base station, and further the bi-directional communication unit modulates the processed digitized results received from the plurality of digital electronic blocks into the alternating electric field to be demodulated from the base station convertor;
a second electrode for closing the circuit with the base station, the user and earth's ground.

2. The device according to claim 1, wherein the PolyMonIC chip further comprises a plurality of optoelectronic blocks arranged to perform measurements of the vital signs of the user using optical effects.

3. The device according to claim 2 wherein the optoelectronic blocks are connected to the analog electronic blocks and the digital electronic blocks for further processing of the optical effects under the control of the digital unit.

4. The device according to claim 3 wherein the plurality of electronic switches controls the operation of the optoelectronic blocks under the control of the digital unit.

5. The device according to claim 1 wherein the PolyMonIC chip further comprising a plurality of piezo rings arranged inside the polymer patch to convert mechanical vital signs into electrical signals capactively coupled to and received from the bifilar electrode.

6. The device according to claim 5 further wherein the plurality of piezo rings arranged inside the polymer patch convert electrical pulses from the PolyMonIC chip into mechanical signals to stimulate the user.

7. The device according to claim 1 wherein the PolyMonIC chip further comprising an electronic Ultra-Turing machine to alter the sequence in the digital unit according to digitized calculation results of the processed vital signs to optimize the performance of the digital unit.

8. The device according to claim 1 wherein the PolyMonIC chip further comprising a superposition unit containing a Pascal triangle structure for calculating a dynamic baseline for drift compensation and probabilities to classify the received processed vital signs.

9. The device according to claim 1 wherein the PolyMonIC chip further comprises a unique identifier to generate a unique identification code to be received by the base station on demand modulated by the bi-directional communication unit and emitted by the second electrode.

10. The device according to claim 1 wherein the PolyMonIC chip further comprises a plurality of micro-needles arranged within the polymer patch to penetrate the human skin.

11. The device according to claim 10 wherein the PolyMonIC chip further comprises a plurality of ionizer circuits arranged to perform positive and/or negative charges under the control of the digital unit on at least one of: the bifilar electrode, and the plurality of micro-needles.

12. The device according to claim 10 wherein the PolyMonIC chip further comprises a plurality of reservoirs arranged to store bio-chemical substances to be released into the user through the micro-needles.

13. The device according to claim 12 wherein the plurality of reservoirs arranged to store bio-chemical substances to be released into the user through the micro-needles under the control of the ionizer circuits.

14. The device according to claim 1 wherein the base station further comprises an interface for communicating processed data received from the controller over communication networks, further the interface receives instructions over the communication network to be communicated with the controller.

15. The device according to claim 10 wherein the plurality of micro-needles are bio-needles that dissolve in the user's skin.

16. The device according to claim 1 wherein the PolyMonic chip further comprises an optical unit attached to the polymer patch to generate signals based on vital conditions of the user.

17. The device according to claim 14 wherein the interface is communicating with interfaces of other base stations from other polymer patches to share vital sign data among users.

18. The device according to claim 2 wherein the PolyMonIC chip further comprises a pinhole to guide the optical effects to and from the optoelectronic blocks.
